(19) United States
(12) Patent Application Publication
(10) Pub. No.: US 2008/0009936 A1
(43) Pub. Date: Jan. 10, 2008

(54) STENT DEVICE FOR ANASTOMOSES OF BLOOD VESSELS AND OTHER TUBULAR ORGANS

(75) Inventors: John Y. S. KIM, Chicago, IL (US); Artur Kaganovsky, Wheeling, IL (US); Michael Henry, Wheeling, IL (US)

Correspondence Address: DRINKER BIDDLE & REATH LLP;ATTN: PATENT DOCKET DEPT., 191 N. WACKER DRIVE, SUITE 3700, CHICAGO, IL, 60606, US

(73) Assignee: Eidosmed, LLC, Chicago, IL (US)

(21) Appl. No.: 11/744,733

(22) Filed: May 4, 2007

Related U.S. Application Data
(60) Provisional application No. 60/797,946, filed on May 5, 2006.

(51) Int. Cl. A61F 2/06

(52) U.S. Cl. 623/1.15

(57) ABSTRACT

A method for connecting two blood vessels or other tubular organs in end-to-end fashion and a stent therefor including a central portion that may be expanded, nipples at opposite ends of the central portion and a lumen passing through the central portion and the nipples. The nipples may be of different cross-sectional sizes or funnel-shaped to enable the stent to be used in attaching two vessels or organs of different diameters.

### CROSS-REFERENCE TO RELATED PATENT APPLICATIONS

This patent application claims the benefit of U.S. Provisional Patent Application No. 60/797,946, filed May 5, 2006.

### FIELD OF THE INVENTION

The present invention relates generally to devices for connecting the ends of interrupted blood vessels, urethras or other tubular organs and, more particularly, to a stent that is secured in place within adjacent ends of two interrupted blood vessels, urethras or other tubular organs to establish flow therethrough.

### BACKGROUND OF THE INVENTION

Vascular and microvascular surgery entails the connection of the ends of various sizes and types of interrupted blood vessels. In all cases, it is essential to obtain a secure and leak-free connection between the blood vessel ends.

Currently, both vascular and microvascular surgery utilize extremely technically-demanding processes of hand sewing vessel ends to suture them together. Often, in order to insure successful outcomes, this must be done under loupe or microscopic magnification using delicate suturing techniques requiring great skill and experience on the part of the surgeon. Furthermore, despite surgeons' best efforts and initially successful suturing, the resulting anastomotic site is subject to thrombosis which may adversely impact patient outcomes. There is also the danger that the stitches will cut through a vessel which can produce leakage and failure of the suture. Other drawbacks in connection with the use of hand sewing are the slow and tedious process that it entails which often makes for operations of long duration, and the possible presence of suture material in the lumen of the vessel which may interfere with blood flow or form a nidus for thrombus formation. Additionally, the time required for performing such painstaking microsurgery is significant and increased ischemia time during which time the vessels are not connected puts local tissue—and ultimately the patient—at risk.

There is currently no widely accepted stitchless device or technique for simply and reliably joining arteries end-to-end. Known devices for coupling veins end-to-end without stitching typically involve affixation of separate collar-like members to each of the vessel ends and joinder of these members. However, the transposition of these vein couplers to arteries is difficult because the arterial wall thickness is greater than that of veins. This prevents the proper folding of the artery necessary for proper execution of the coupling device. Also, the collars interfere with or prevent direct contact between the two vessel ends. Additionally, the affixation and joinder techniques currently in use with vein couplers are generally cumbersome and technically difficult to use.

Also, it is often necessary to attach blood vessels of different diameters end-to-end. This mismatch of vessel diameters can create mechanical problems at the anastomotic site that predisposes the site to flow irregularities and thrombosis. Current coupling devices do not optimally address significant vessel mismatches.

In view of the limitations and shortcomings of current hand sewn suturing techniques and stitchless vein attachment devices, it would be highly desirable to provide a simple, reliable device and method that can be used to join both veins or thicker walled arteries end-to-end, quickly, efficiently, securely, and without danger of damaging vessel walls, inducing thromboses, or causing other complications. If a range of different vessel diameters could be accommodated by a single device and method, this would be a further important contribution to the art. If the device and method made possible quick vascular diameter matching and alignment, minimized anastomotic leakage and kinking, and facilitated blood flow at the anastomosis site to minimize the chance of clotting, an important advance in the art of joining blood vessels end-to-end would be at hand. The present invention embodies a device and method that provides all of these properties and advantages. The present invention also provides like properties and advantages when used to join other tubular organs like urethras.

Further advantages and characteristics of the present invention will become apparent to those skilled in the art from the detailed description that follows and the accompanying drawings. Preferred but nonexclusive embodiments of the invention are illustrated and discussed by way of non-limiting examples of the invention.

### BRIEF SUMMARY OF THE INVENTION

The present invention may be used wherever two blood vessels or other tubular organs like urethras must be connected in an end-to-end fashion. Common applications include, for example, attachment of blood vessels for organ transplantation, free tissue transfers, or various forms of cardiovascular bypass surgery, as well as urethral anastomoses. The invention is able to accommodate a range of vessel and organ sizes in these and other applications.

The present invention thus consists of stents for connecting two blood vessels or other tubular organs in end-to-end fashion to enable flow therethrough. The invention also includes methods of using such a device in achieving anastomoses of blood vessels or other tubular organs.

The stent includes a central portion, inlet and outlet nipples at opposite ends of the central portion and a lumen passing through the central portion and the nipples. The central portion preferably will be enlarged relative to the nipples to facilitate flow through the stent. Indeed, it is preferred that such an enlarged central portion will have a maximum cross-section between about 125 to 200% of the diameter of the openings of the nipples. Also, the stent preferably will have an outer surface configured so that the transition from the outer surface of the nipples to the outer surface of the central expanded portion is gently sloped.

The contours of the lumen in one embodiment will generally follow the contours of the outer surface of the stent. In embodiments in which the stent has an expanded central portion, the stent wall may be thickened in the area of the expanded portion to reduce the cross-section of the lumen in that area to varying degrees up to and including to such an extent that the inner wall will be flat and the lumen through the stent will be of a uniform tubular shape.

The stent may be made of a solid material or it may be made of a porous mesh. Preferably, the outer surface of the stent will be coated with a tissue ingrowth material. In order to improve the purchase between the stent and the interior of the portions of the vessels that will overlie the stent, the outer surface may also have one or more of a raised netting surface, hooks, serrations, barbs, ribs or a surface configured for ingrowth from the vessels attached to the stent.

The invention also entails a method of joining the ends of two blood vessels (or other tubular vessels) using a stent as discussed above. In practicing the method, the nipples of the stent will be inserted within the lumens of the vessels and the vessels will be clamped to the stent. In stents having a solid wall, one or both of the vessels may be drawn only partially up the outer wall of the nipples and then clamped or the vessels may be drawn up to approximately the midpoint of the stent so that their edges meet. When the stent is in the form of an open mesh, the blood vessels must be drawn up over the entirety of the stent to prevent leakage therethrough. Finally, clamps will be applied as needed either at the nipples and/or at locations on the central portion of the stent. In one embodiment, the two vessels be drawn up along the outer surface of the stent until the vessel ends meet and a clamp applied over the interface between the adjoining vessel ends to both attach the vessels to the stent and to seal the interface between the adjoining vessel ends.

### BRIEF DESCRIPTION OF THE DRAWINGS

Preferred embodiments of the invention are described with reference to the accompanying drawings, in which like elements bear like reference numerals, and wherein:

FIG. 1 is a perspective view of one embodiment of a stent in accordance with the present invention;

FIG. 1A is a cross-sectional views of the stent of FIG. 1 taken along lines 1A-1A;

FIG. 2 is a perspective view of another embodiment of a stent in accordance with the present invention in which the two stent nipples accommodate different vessel lumen sizes;

FIG. 3 is yet another embodiment of the invention in which the stent accommodates a range of different vessel lumen sizes;

FIG. 4 is a diagrammatic representation of the outer surface of the stents in accordance with the present invention in which expanded central portions of the stent are successively reduced;

FIGS. 5A-5C are diagrammatic representations of stents with outer surfaces corresponding to the stent depicted in FIG. 1 in which the shapes of the lumens of the stents are of successively reduced curvatures;

FIGS. 6A-6F are partial views of stents in accordance with the present invention in which the outer surfaces of the stents have been configured in different ways to improve the purchase between the stent and the interior of vessels attached to the stent;

FIGS. 7A-7C are views of clips and clamps that may be used in accordance with the present invention to fix vessels to the stents of FIGS. 1-3;

FIG. 7D is a perspective view of a crimp clamp and associated crimping tool that may be used in accordance with the present invention to fix vessels to the stents of FIGS. 1-3; and

FIGS. 8A-8D are diagrammatic views illustrating the use of a stent of the present invention to attach a pair of vessel ends in accordance with the present invention.

### DETAILED DESCRIPTION OF PREFERRED EMBODIMENTS

The following detailed descriptions and accompanying drawings are provided for the purpose of illustrating and describing certain presently preferred embodiments of the invention. These descriptions and drawings are not intended to limit the scope of the protection of the invention in any way.

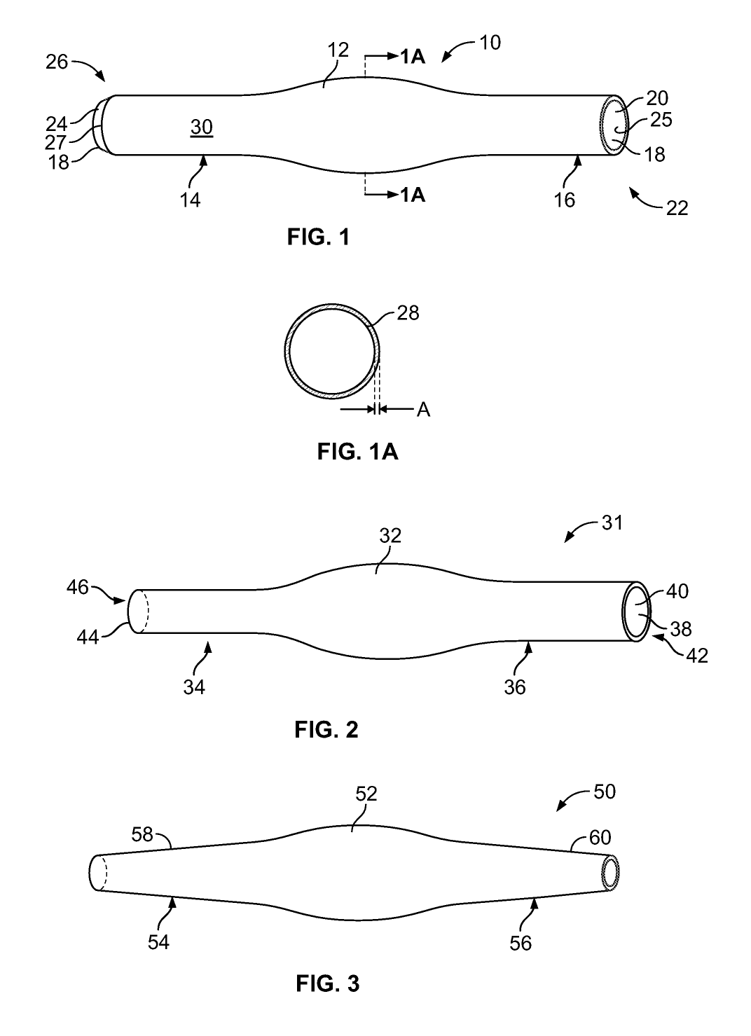

Turning to FIG. 1, a stent 10 in accordance with one embodiment of the invention is illustrated. Stent 10 includes a central expanded portion 12 with inlet and outlet nipples 14 and 16 at opposite ends of the central expanded portion. Although the central expanded portion is generally at the midpoint of the length of the stent as illustrated in FIG. 1, the expanded portion need not be at the midpoint of the device. Nipples 14 and 16, which will enter the lumens of the vessels being joined, may be elongated as shown to prevent kinking or tethering in proximity to the anastomosis. As will be explained further below, the transition from the nipples to the central expanded portion of the stent is gently sloped to help dilate and lift the vessels over the stent, producing expansion of the vessels to minimize the danger of thrombosis at the point of anastomosis.

A lumen 18 passes through the central expanded portion and the nipples of the stent from a first generally annular nipple opening 20 in end 22 of the stent through the center expanded portion 12 to a second opening 24 at end 26. Ends 22 and 26 may be chamfered as shown at 27 to facilitate entry of the nipple into the vessel lumens. The leading edge of the nipple, however, should be rounded to minimize the danger of damaging the vessel as it moves along the nipple lumen. The walls 28 of the stent are generally of uniform thickness so that the contours of lumen 18 generally follow the contours of the outer surface 30 of the stent. Also, the surface 25 of lumen 18 preferably is smooth in order to maximize laminar flow and minimize the danger of clot formation. Indeed, to facilitate blood flow and further minimize the danger of clot formation, the surface of the lumen may be coated with anti-thrombogenic materials including endothelial cells, fibrous coatings, or other compositions. The coating compositions can be growth and differentiation modulators, such as but not limited to, TGF-β and/or bone morphogenic protein(s) (BMPs), bFGF, IGF-1, IGF-II, and anticoagulants, such as but not limited to, streptokinase, urokinase (uPA), and IPA, or ascorbic acid.

As seen in FIG. 1, central expanded portion 12 has an enlarged cross-section with respect to nipple openings 20 and 24. It is preferred that the maximum cross-section of the enlarged central portion be between about 125% to 200% of the diameter of the stent at the nipple openings.

The stent may be formed from an appropriate metal such as stainless steel, or from a shape memory alloy like Nitinol. If made of metal, the stent can be laser-machined or it can be mechanically expanded in the center portion. The stent may have a solid wall or it may be made of a porous mesh. Also, the outer surface of the stent may be coated with a membrane of porous ePTFE or another polymer to promote tissue ingrowth to help prevent leaking.

In an alternative embodiment, the stent may be formed from a polymer. In a preferred embodiment the polymer may be selected from the group consisting of shape memory polymers, silicone, polyurethane, polyethylene, acrylonitrile butadiene stryrene (ABS), polycarbonate, polypropylene, styrene, polyamide (nylon), polymide, PEEK, PEBAX, polyester, PVC, fluropolymers (TEFLON), and co-polymers.

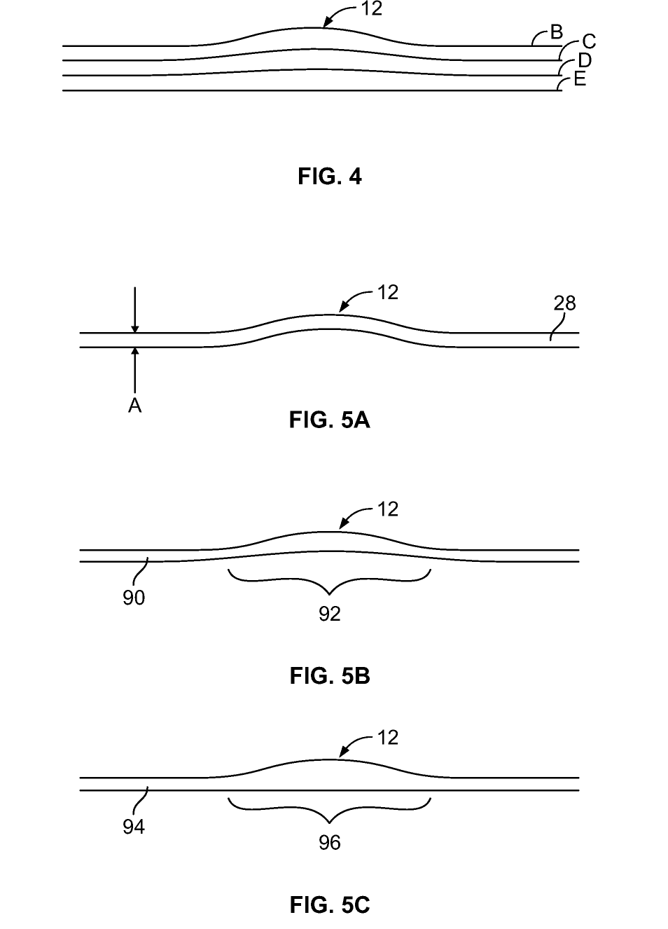

In another embodiment of the invention as illustrated in FIG. 2, a stent 31 with a central expanded portion 32, nipples 34 and 36, a lumen 38, and respective openings 40 and 44 at stent ends 42 and 46 is shown. In this embodiment, however, nipple 36 is larger in outer diameter than nipple 34 to enable the stent to be used in attaching two vessel ends of different diameters.

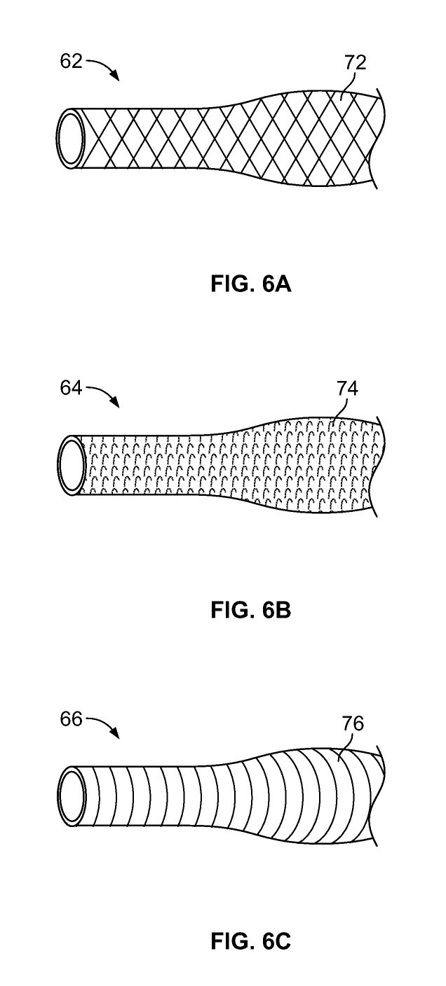

In yet another embodiment of the invention illustrated in FIG. 3 stent 50 is designed to accommodate a range of different vessel sizes. As shown in this figure, the stent includes a central expanded portion 52 with nipples 54 and 56 at opposite ends of the central expanded portion. In this embodiment, however, the nipples have downwardly ramping walls 58 and 60 which form funnel-like structures with diameters that increase from the distal ends of the nipples of the stent toward the central expanded portion. These funnel-like structures can accommodate different vessel diameters as will be explained in greater detail below.

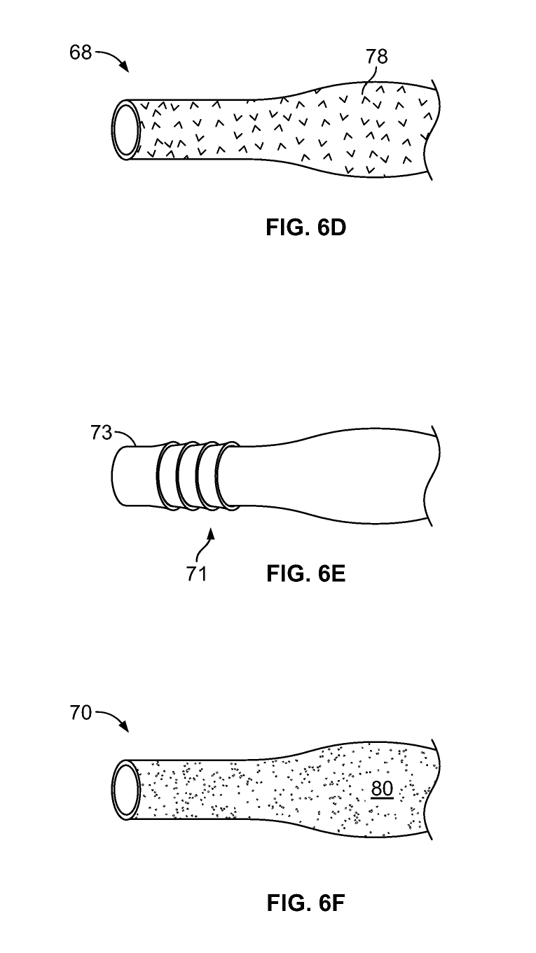

FIG. 4 represents diagrammatically a partial cross-section of the outer wall of a series of stents in accordance with the present invention in which the degree of curvature at the outer surface of the expanded central portion ranges from that shown in the stent of FIGS. 1-3 (depicted as “B”), to intermediate degrees of curvature (depicted as “C” and “D”) to a straight or tubular outer surface in which there is no expanded portion (depicted “E”). The decreasing outer curvatures will produce decreasing vessel dilation when the stents are used to join vessel ends.

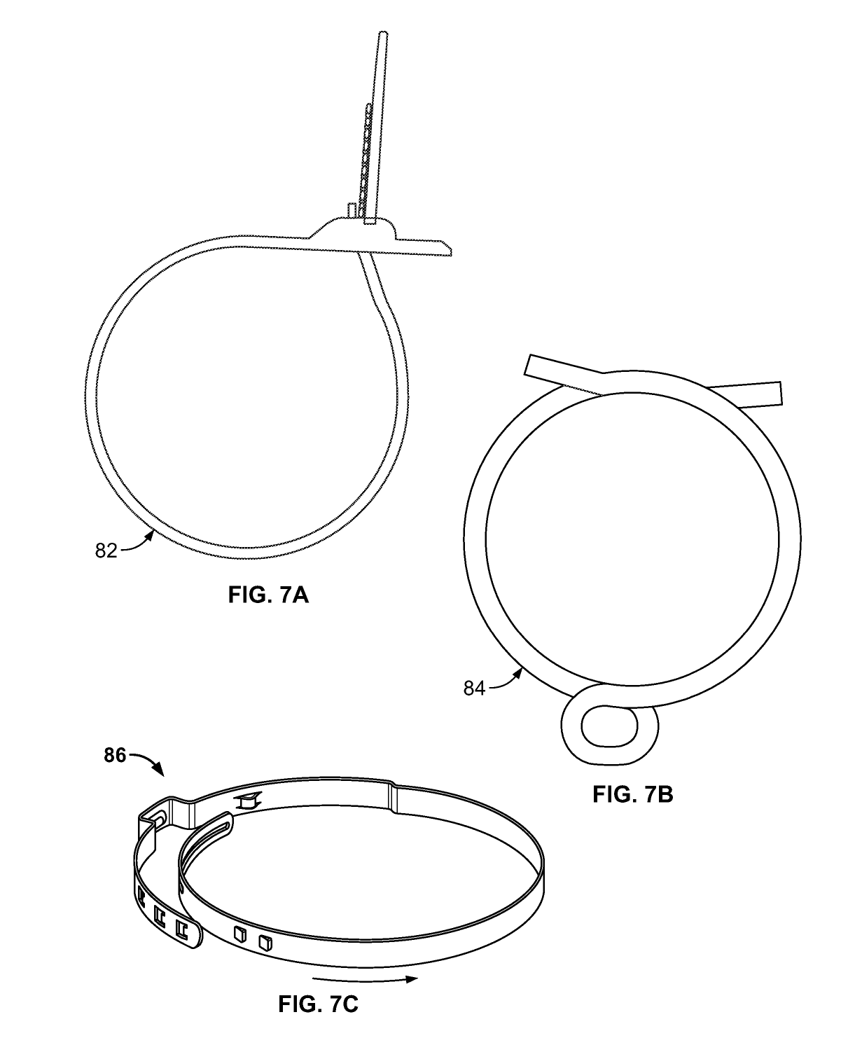

As noted above, in the stent 10 of FIG. 1, wall 28 is of generally uniform thickness. The thickness of wall 28 is depicted as “A” in FIGS. 1A and 5A. However, in alternate embodiments of the invention, the wall may be thickened in the area of the expanded central portion to reduce the cross-section of the lumen of the device in the area of the expanded central portion of the stent. Thus, in the embodiment of FIG. 5B, wall 90 is thickened at 92 to reduce the cross-section of the lumen of the device in the area of the expanded portion of the stent so that the lumen cross-section is only slightly enlarged relative to the diameter of the lumen at the nipples. In the embodiment of FIG. 5C, wall 94 is thickened at 96 so that the lumen cross-section is generally equal to the diameter of the lumen at the nipples creating a uniform tubular lumen through the stent.

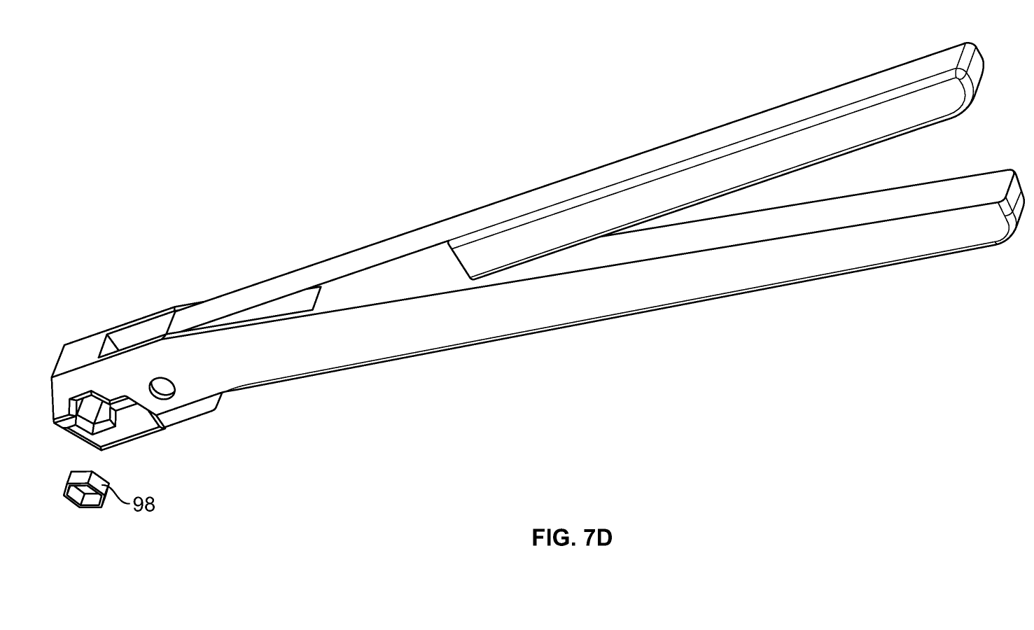

FIGS. 6A-6E are partial views of alternative outer surfaces of nipples 62, 64, 66, 68, 70 and 71 of a stent in accordance with the present invention. All or portions of the surfaces of these nipples are configured, respectively, with a raised line netting surface 72, hooks 74 like those used in velcro attachments, a series of serrations 76, barbs 78, ribs 73 and a porous ingrowth surface 80 to improve the purchase between the stent and the vessel interior which overlies the nipple surfaces. Additionally, it is noted that ribs 73 of FIG. 6E may increase in diameter from the distal end of the nipple to further enhance the attachment to an overlying vessel. In yet another alternative embodiment, the nipple surface will be roughened or otherwise treated to maximize adhesion where an adherent (e.g., fibrin sealant or another appropriate tissue-based adhesive or glue) is used to improve the seal between the vessels and the stent. In all cases, these configurations of the stent outer surfaces may cover the entirety of the surface of the stent or only portions thereof (e.g., only the nipples, only the leading portions of the nipples, or only the central section).

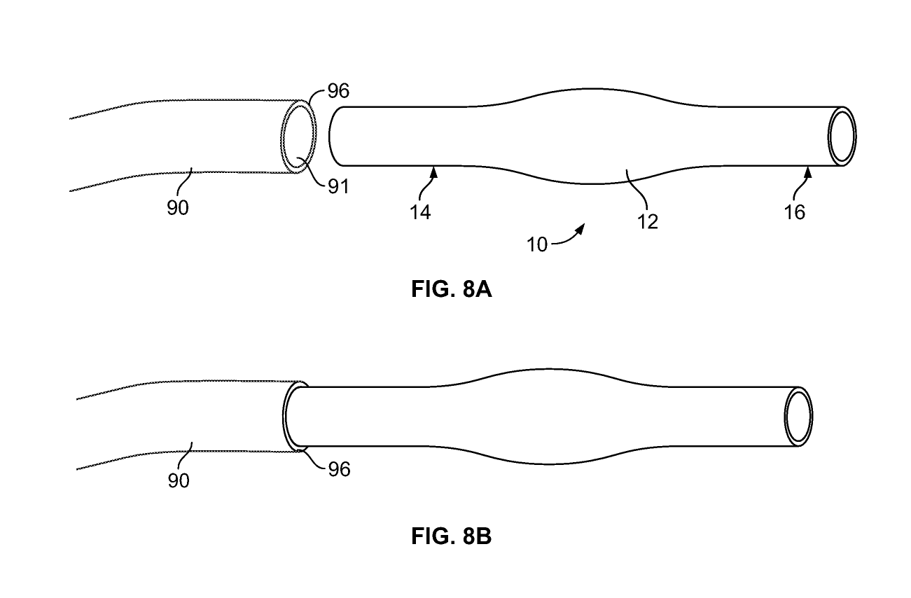

In order to join vessels by way of the stent of the invention, clamps will generally be placed over portions of the vessels overlying the stent. Any appropriate spring clip, cable clamp, hose clamp design, or band. For example, adjustable cable tie 82, spring clip 84, or cable clamp 86 of FIGS. 7A-7C could be used. Alternatively a crimpable band 88 could be applied using a crimping tool like tool 89 (FIG. 7D). An appropriate elastic band, a “C” shaped retaining clip, or a suture could also be used. Preferably, the clips, clamps, bands, etc. will have appropriate dimensions and stops to insure that excessive pressure is not applied when they are closed over a vessel to fix it to the stent. The clips, clamps or bands may be made, for example, of stainless steel, titanium, or other acceptable metal or they may be made of an acceptable polymer such as silicone, polyurethane, polyethylene, acrylonitrile butadiene stryrene (ABS), polycarbonate, polypropylene, styrene, polyamide (nylon), polyimide, PEEK, PEBAX, polyester, PVC, fluropolymers (TEFLON), and available co-polymers thereof. Also, as noted above, the stent may be made of a thermomodulated material (metal or polymer) that will expand due to the warm blood that flows through it after the stent and clips, clamps, etc. are in place to form a tighter seal against the clamps after placement. Conversely, the clips or clamps may be made of a thermomodulated material which contracts to form a tighter sea against the vessel wall and the stent as the warm blood flows through the stent following placement.

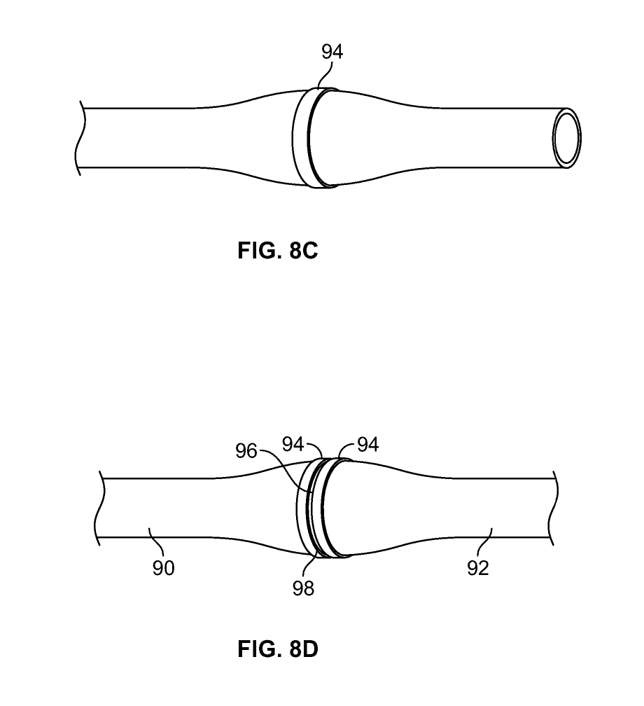

The following examples illustrate modes of practicing the method of the invention but should not be construed as in any way limiting its scope. A stent in accordance with the present invention thus may be used as illustrated in FIGS. 8A-8D to join blood vessels end-to-end, for example, as follows:

### EXAMPLE 1

1. Two blood vessels 90 ad 92 (FIGS. 8A and 8D) with ends of like diameter will be prepared for attachment using known surgical techniques.
2. A stent 10 will be chosen with nipples 14 and 16 having an outer diameter generally corresponding to the lumens of blood vessels 90 and 92. In an alternative embodiment, the nipples will have an outer diameter slightly greater than the native vessel diameter to produce dilation at the vessel ends.
3. The first nipple 14 of the stent will be placed within lumen 91 of the first vessel 90 (FIG. 8B) and drawn up onto the stent to approximately the midpoint of enlarged center portion 12, as shown in FIG. 8C. The center-expanded design of the stent thus causes the vessel to drape in expanded form over the stent, providing dilation of the vessel at the point of anastomosis, which minimizes the likelihood of thrombosis and clot formation.
4. A clamp 94 (illustrated diagrammatically) will then be deployed as shown in FIG. 8C to affix the end of the first vessel to the enlarged center portion. Although the clamp is shown in these Figures as attached near the midpoint of enlarged portion 12, it may be attached elsewhere such as along nipple 14. Also, clamp application may be delayed until both vessels are positioned on the stent and, if desired, a single clamp may be used to simultaneously seal the interface between abutting vessel ends and fix the vessels to the stent.

Also, it should be appreciated that when the stent has porous mesh walls, the entire stent must be covered with the clamped-in-place vessel ends. However, when the stent has solid walls, one or both of the vessels need not be drawn up to the midpoint of the enlarged center portion but rather may be clamped in place with its end at a desired position on stent nipple.
5. The same procedure is followed with respect to the second vessel and the second nipple of the stent producing a configuration, as illustrated in FIG. 6D.
6. Once the two vessels are clamped in place with their ends 96 and 98 abutting generally over the midpoint of the enlarged center portion of the stent, blood flow will commence through the vessels and across the stent so that the anastomosis can be tested and adjusted as required.

### EXAMPLE 2

The procedure of Example 1 is followed except that vessels with different diameter lumens are joined using the stent of FIG. 2.

### EXAMPLE 3

The procedure of Example 1 is followed except that vessels with different diameter lumens are joined using the stent of FIG. 3. Additionally, in this case the vessels are too fragile to be pulled to the midpoint of the enlarged center portion of the solid-walled stent being used and so are only pulled up onto ramping walls 58 and 60 an appropriate distance and clamped in place there.

### EXAMPLE 4

In this example, the outer surfaces of the stents are configured as in one of FIGS. 6A-6E to improve the purchase once the clamp is deployed and/or tissue ingrowth surfaces as in FIG. 6F are provided to improve the purchase between the vessel and the stent over time. Additionally, growth factor may be applied to accelerate the ingrowth process. Finally, in addition or in the alternative, an adherent may be applied to the surface of the stent before the vessel is positioned on the stent to provide an enhanced seal and greater reliability of the anastomosis.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. It should be understood that the illustrated embodiments are exemplary only, and should not be taken as limiting the scope of the invention.

What is claimed is:

1. A stent for connecting two blood vessels or other tubular organs in end-to-end fashion to enable flow therethrough comprising:
a central portion;
inlet and outlet nipples at opposite ends of the central portion; and
a lumen passing through the central portion and the nipples.

2. The stent of claim 1 in which the central portion is expanded relative to the nipples.

3. The stent of claim 1 in which the nipples are elongated.

4. The stent of claim 2 in which the end of at least one nipple is chamfered to facilitate entry of the nipple into a vessel lumen.

5. The stent of claim 2 in which the maximum cross-section of the enlarged central portion is between about 125 to 200% of the diameter of the stent at the nipple openings.

6. The stent of claim 2 in which the stent has an outer surface and the transition from the outer surface of the nipples to the outer surface of the central expanded portion is gently sloped.

7. The stent of claim 2 in which the contours of the lumen generally follows the contours of the outer surface of the stent.

8. The stent of claim 1 in which the lumen is smooth to maximize laminar flow and minimize the danger of clot formation.

9. The stent of claim 1 in which the lumen is coated with an anti-thrombogenic material.

10. The stent of claim 1 in which the stent is made of a porous mesh.

11. The stent of claim 1 in which the stent has an outer surface and the outer surface of the stent is coated with a tissue ingrowth material.

12. The stent of claim 1 in which the nipples are of different sizes to enable the stent to be used in attaching two vessels or organs of different sizes.

13. The stent of claim 2 in which at least one of the nipples has a funnel-like structure with a diameter that increases from the distal end of the nipple toward the central portion of the stent.

14. The stent of claim 2 in which the stent is thickened in the area of the expanded portion of the stent to reduce the cross-section of the lumen in the area of the expanded portion of the stent.

15. The stent of claim 2 in which the lumen is of a uniform tubular shape.

16. The stent of claim 1 in which portions of the outer surface of the stent have a configuration for improving purchase between the stent and the interior of a vessel which overlies the stent chosen from the group consisting of: a raised netting surface, hooks, serrations, barbs, ribs, and an ingrowth surface.

17. The stent of claim 1 in which the stent is made from a shape memory material.

18. A blood vessel anastamosis comprising:
a stent for connecting two blood vessels in end-to-end fashion to enable flow therethrough, the stent having a central portion, nipples at opposite ends of the central portion, and a lumen passing through the central portion and the nipples; and
two blood vessels attached to the stent.

19. The blood vessel anastamosis of claim 18 in which the central portion of the stent is expanded relative to the nipples.

20. The blood vessel anastamosis of claim 18 in which each of the stent nipples and the blood vessels are of different corresponding sizes.

21. The blood vessel anastamosis of claim 18 in which at least one of the nipples of the stent has a funnel-like structure with a diameter that increases from the distal end of the nipple toward the central portion of the stent.

22. A blood vessel anastamosis of claim 18 in which the blood vessels are clamped to the stent.

23. A method of joining two blood vessels or other tubular organs end-to-end comprising:
preparing the vessel or organ ends for attachment;
providing a stent for connecting the vessel or organ ends for attachment to enable flow therethrough, the stent having a central portion, inlet and outlet nipples at opposite ends of the central portion, and a lumen passing through the central portion and the nipples;
placing a first nipple of the stent within the lumen of the first vessel or organ;
placing the second nipple of the stent within the lumen of the second vessel or organ; and
clamping the blood vessel or organs to the stent.

24. The method of claim 23 in which the vessels or organs are drawn up along the stent so that they abut at approximately the midpoint of the stent central portion.

25. The method of claim 24 in which a clamp is placed over the vessels or organs where they abut to both seal the interface therebetween and fix the vessels or organs to the stent.
